(19) United States
(12) Patent Application Publication
(10) Pub. No.: US 2026/0041928 A1
(43) Pub. Date: Feb. 12, 2026

(54) BEAUTY INSTRUMENT AND CONTROL METHOD THEREFOR

(71) Applicant: Shenzhen Ulike Smart Electronics Co., Ltd., Shenzhen (CN)

(72) Inventor: DI ZENG, Shenzhen (CN)

(21) Appl. No.: 19/141,339

(22) PCT Filed: Aug. 21, 2024
(86) PCT No.: PCT/CN2024/113579

§ 371 Date: Jun. 20, 2025

(30) Foreign Application Priority Data
Dec. 4, 2023 (CN) 202311665127.0

(51) Int. Cl. A61N 5/06; A61F 7/00; A61F 7/02; A61N 5/00

(52) U.S. Cl. CPC A61N 5/0616; A61F 7/02; A61F 2007/0052; A61N 2005/007; A61N 2005/0626; A61N 2005/0663

(57) ABSTRACT

A control method for a beauty instrument and a beauty instrument, including: obtaining an Nth light irradiating mode configured for the beauty instrument; setting a light irradiator to irradiate pulse light N times and a preset threshold corresponding to each time of irradiation of the pulse light, where N is a positive integer greater than or equal to 1; charging an energy storager, and controlling, when the energy storager is charged to a first preset threshold and the light irradiator is triggered, the light irradiator to irradiate first pulse light; and determining whether N times of irradiation of the pulse light have been completed, if no, continuing to charge the energy storager, and controlling, when the energy storager is charged to a preset threshold corresponding to a next time of irradiation of the pulse light and the light irradiator is triggered, the light irradiator to irradiate next pulse light.

### CROSS REFERENCE TO RELATED APPLICATIONS

This application is based on Chinese Patent Application No. 202311665127.0, filed on Dec. 4, 2023, and entitled “BEAUTY INSTRUMENT AND CONTROL METHOD THEREFOR”, and claims priority to the Chinese Patent Application, which is incorporated herein by reference in its entirety.

### TECHNICAL FIELD

Embodiments of this application relate to the field of beauty and skincare technologies, and in particular, to a beauty instrument and a control method therefor.

### BACKGROUND

In related technology, beauty instruments can irradiate specific light to human skin by using a light irradiator, and the light acts on the skin to provide caring for the skin. Current beauty instruments are limited by factors such as a body and hardware settings, and usually operate in a single pulse mode, that is, the beauty instruments are controlled to irradiate light in a single pulse to implement a specific skin caring function. However, as a user's requirement for beauty and skincare increases, there is still room to enhance richness of function settings of the current beauty instruments.

### SUMMARY

In view of the foregoing content, it is necessary to provide a control method for a beauty instrument and a beauty instrument, so that more function modes can be configured to improve function richness of the beauty instrument.

According to a first aspect, an embodiment of this application provides a control method for a beauty instrument, where the beauty instrument includes an energy storager and a light irradiator, the energy storager is configured to be charged and perform discharging, and the light irradiator is configured to irradiate pulse light based on power supply of the energy storager, and the method includes: obtaining an Nth light irradiating mode configured for the beauty instrument; setting, based on the Nth light irradiating mode, the light irradiator to irradiate the pulse light N times and a preset threshold corresponding to each time of irradiation of the pulse light, where N is a positive integer greater than or equal to 1; charging the energy storager, and controlling, when it is determined that the energy storager is charged to a first preset threshold in the Nth light irradiating mode and the light irradiator is triggered, the light irradiator to irradiate first pulse light; determining whether N times of irradiation of the pulse light have been completed, if no, continuing to charge the energy storager, and controlling, when it is determined that the energy storager is charged to a preset threshold corresponding to a next time of irradiation of the pulse light and the light irradiator is triggered, the light irradiator to irradiate next pulse light; and repeatedly performing the step of determining whether N times of irradiation of the pulse light have been completed.

According to the control method for a beauty instrument in this solution, the energy storager is charged at different stages. Therefore, the light irradiator can irradiate the pulse light a plurality of times based on charging at different stages, so that the beauty instrument can provide various care manners for human skin in a plurality of light irradiating manners. This can meet a user's requirement for different functions of beauty and skincare, and improve function richness of the beauty instrument.

In some implementations, the preset threshold is a pulse voltage threshold or a charging time threshold.

In some implementations, the Nth light irradiating mode further includes a pulse light energy level, and the method further includes: obtaining the pulse light energy level in the Nth light irradiating mode configured for the beauty instrument; and setting, based on the pulse light energy level, the first preset threshold corresponding to irradiation of the first pulse light to an Nth preset threshold corresponding to irradiation of Nth pulse light.

In some implementations, when N is greater than or equal to three, durations of time intervals between consecutive irradiations of the pulse light are different in the Nth light irradiating mode.

In some implementations, the method further includes: setting a first pulse width of the first pulse light to an Nth pulse width of Nth pulse light in the Nth light irradiating mode, where the pulse width is used to represent a duration in which the light irradiator irradiates the pulse light.

In some implementations, the beauty instrument further includes a cooler, and the method further includes: when an ice compress mode is configured for the beauty instrument, enabling the cooler to work at first power.

In some implementations, the method further includes: when the light irradiating mode is configured for the beauty instrument, enabling the cooler to work at second power, where the second power is less than the first power.

In some implementations, the beauty instrument further includes a light modulator, configured to irradiate light of different colors, and the method further includes: in the Nth light irradiating mode, controlling the light modulator to respectively irradiate light of a first color to light of an Nth color when the first pulse light to Nth pulse light are irradiated; or the beauty instrument further includes a cooler and a light modulator configured to irradiate light of different colors, and the method further includes: when an ice compress mode is configured for the beauty instrument, controlling the light modulator to irradiate light of an (N+1)th color, where the first color, the Nth color, and the (N+1)th are different.

In some implementations, the beauty instrument further includes an indicator, and the method further includes: when the Nth light irradiating mode or an ice compress mode is configured for the beauty instrument, enabling the indicator to separately indicate different modes.

In some implementations, the beauty instrument further includes a distance detector and a trigger button of the light irradiator; the controlling, when it is determined that the energy storager is charged to a first preset threshold in the Nth light irradiating mode and the light irradiator is triggered, the light irradiator to irradiate first pulse light includes: controlling, when it is determined that the energy storager is charged to the first preset threshold in the Nth light irradiating mode, the distance detector of the beauty instrument detects a preset distance threshold, and the trigger button is triggered, the light irradiator to irradiate the first pulse light; and the controlling, when it is determined that the energy storager is charged to a preset threshold corresponding to a next time of irradiation of the pulse light and the light irradiator is triggered, the light irradiator to irradiate next pulse light includes: controlling, when it is determined that the energy storager is charged to the preset threshold corresponding to the next time of irradiation of the pulse light and the distance detector of the beauty instrument detects the preset distance threshold, the light irradiator to irradiate the next pulse light.

According to a second aspect, an embodiment of this application provides a beauty instrument, including a controller, where the controller is configured to perform the foregoing control method.

### BRIEF DESCRIPTION OF DRAWINGS

FIG. 1 is a schematic diagram of function modules of a beauty instrument according to an embodiment of this application;

FIG. 2 is a flowchart of a control method for a beauty instrument according to an embodiment of this application;

FIG. 3 is a schematic diagram of function modules of a beauty instrument according to another embodiment of this application;

FIG. 4 is a flowchart of a control method for a beauty instrument according to another embodiment of this application; and

FIG. 5 is a flowchart of a control method for a beauty instrument according to another embodiment of this application.

Symbol description of main elements

| | | | |
|---|---|---|---|
| beauty instrument | 100 | energy storager | 10 |
| light irradiator | 20 | distance detector | 22 |
| trigger button | 24 | controller | 30 |
| cooler | 40 | light modulator | 50 |
| heat dissipator | 60 | indicator | 70 |

This application is further described in the following DESCRIPTION OF EMBODIMENTS with reference to the foregoing accompanying drawings.

### DESCRIPTION OF EMBODIMENTS

In the following, terms “first” and “second” are used only for description purposes, and cannot be understood as an indication or an implication of relative importance or an implicit indication of a quantity of indicated technical features. Therefore, the features defined with “first” and “second” may include one or more of the features explicitly or implicitly. In the description of the embodiments of this application, words such as “example”, “or”, and “for example” are used to represent giving an example, an illustration, or a description. Any embodiment or design solution described as “example” or “for example” in the embodiments of this application should not be explained as being more preferred or having more advantages than another embodiment or design solution. Exactly, use of the words such as “example”, “or”, and “for example” is intended to present a relative concept in a specific manner.

Unless otherwise defined, all technical and scientific terms used in this specification have same meanings as those usually understood by a person skilled in the art of this application. The terms used in the specification of this application are merely used to describe specific embodiments, and are not intended to limit this application. It should be understood that, unless otherwise specified in this application, “/” means “or”. For example, A/B may represent “A or B”. In this application, “and/or” describes only an association relationship for describing associated objects, and represents that three relationships may exist. For example, A and/or B may represent three cases: Only A exists, both A and B exist, and only B exists. “At least one” means one or more. “A plurality of” means two or more. For example, at least one of a, b, or c may represent seven cases: a, b, and c, a and b, a and c, b and c, a, b, and c.

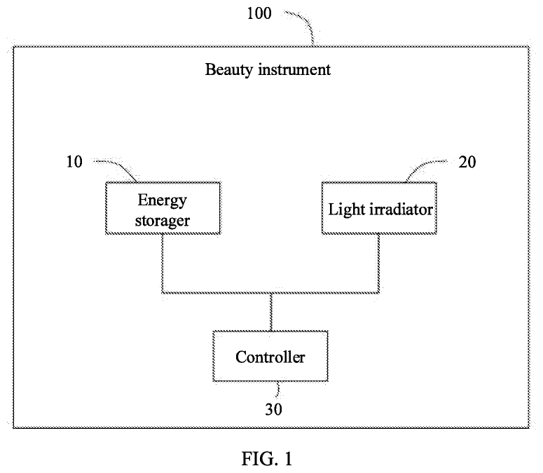

Referring to FIG. 1, an embodiment of this application provides a beauty instrument 100. In some embodiments, the beauty instrument 100 may be configured with a plurality of working modes, and is configured to irradiate preset pulse light one or more times, so that the beauty instrument can provide different care manners for human skin in the plurality of working modes. This can meet a user's requirement for different functions of beauty and skincare, and have good function richness. In some embodiments, the beauty instrument 100 may be but is not limited to an electronic instrument used for beauty and skincare, such as a beauty instrument, a skincare instrument, a hair removal instrument, and a caring instrument.

The beauty instrument 100 includes an energy storager 10, a light irradiator 20, and a controller 30.

The energy storager 10 is configured to store electric energy, be charged, and perform discharging. In some embodiments, the energy storager 10 may be a rechargeable battery or a capacitor.

The light irradiator 20 is connected to the energy storager 10, and the light irradiator 20 is configured to irradiate light based on power supply of the energy storager 10. In some embodiments, the light irradiator 20 may be but is not limited to a xenon lamp or another light-emitting electronic component, and may irradiate preset pulse light by using electric energy released by the energy storager 10. In some embodiments, the light irradiator 20 may irradiate light to skin of a user to perform beauty and skincare treatment on the skin.

The controller 30 is separately connected to the energy storager 10 and the light irradiator 20. The controller 30 is configured to: configure a working mode of the beauty instrument 100, determine a charging status of the energy storager 10, and control the light irradiator 20 to work. In some embodiments, the controller 30 may be but is not limited to a controller or a chip that has a data and information processing function such as a micro controller unit.

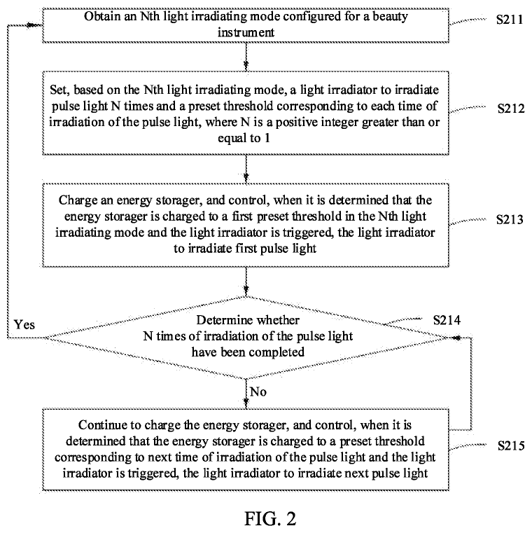

FIG. 2 shows a control method for a beauty instrument according to an embodiment of this application. For example, the control method shown in FIG. 2 may be performed by the beauty instrument 100 shown in FIG. 1, and includes the following steps.

Step S211: Obtain an Nth light irradiating mode configured for the beauty instrument.

In some embodiments, after the beauty instrument 100 is powered on and enters a preset working mode, the controller 30 may obtain the Nth light irradiating mode configured for the beauty instrument 100, and the controller 30 may control the energy storager 10 to be charged. In some embodiments, for a light irradiating mode configured for the beauty instrument 100, the light irradiating mode may include a first light irradiating mode to the Nth light irradiating mode, such as the first light irradiating mode, a second light irradiating mode, . . . , an (N−1)th light irradiating mode, and the Nth light irradiating mode. N is a positive integer greater than or equal to 1. When N is 1, the light irradiating mode may be the first light irradiating mode; when N is 2, the light irradiating mode may be the second light irradiating mode, and so on. In some embodiments, the Nth light irradiating mode may represent that the light irradiator 20 irradiates light N times in the light irradiating mode. It may be understood that, based on a definition of a quantity of times that the light irradiator 20 continuously irradiates light, the first light irradiating mode, the second light irradiating mode, . . . , the (N−1)th light irradiating mode, and the Nth light irradiating mode may also be a single-pulse mode, a dual-pulse mode, a three-pulse mode, . . . , an (N−1)-pulse mode, and an N-pulse mode. In some embodiments, the light irradiating mode may be set by default; in other embodiments, the light irradiating mode may be user-defined, for example, the beauty instrument 100 may be provided with a touch screen, button or other input mode for the user to select or set the light irradiating mode; in still another embodiments, the light irradiating mode may be automatically adjusted by the beauty instrument 100 based on automatic recognition of the user's skin type or condition, which is not limited in this application.

In some embodiments, the energy storager 10 is charged by connecting to an external power supply, or is charged by connecting to an external power supply by using a voltage convertor. This is not limited in this application.

Step S212: Set, based on the Nth light irradiating mode, the light irradiator to irradiate the pulse light N times and a preset threshold corresponding to each time of irradiation of the pulse light, where N is a positive integer greater than or equal to 1.

In some embodiments, the controller 30 may set, based on the obtained Nth light irradiating mode, the light irradiator to irradiate the pulse light N times and the preset threshold corresponding to each time of irradiation of the pulse light. For example, when N is 1, the controller 30 may set, based on the obtained first light irradiating mode, the light irradiator to irradiate the pulse light one time and a first preset threshold corresponding to irradiation of the pulse light. When N is a positive integer greater than or equal to 2, the controller 30 may set, based on the obtained Nth light irradiating mode, the light irradiator to irradiate the pulse light N times and a first preset threshold, a second preset threshold, . . . , an (N−1)th preset threshold, and an Nth preset threshold that correspond to each irradiation of the pulse light. For example, when N is 3, the controller 30 may set, based on the obtained third light irradiating mode, the light irradiator to irradiate the pulse light three times and a first preset threshold, a second preset threshold, and a third preset threshold that correspond to each irradiation of the pulse light. In some embodiments, the preset threshold is a pulse voltage threshold or a charging time threshold. In some embodiments, the first preset threshold to the Nth preset threshold may respectively correspond to a pulse voltage threshold or a charging time threshold to which the energy storager 10 needs to be charged during each time of irradiation of the light among N times of irradiation of light in the Nth light irradiating mode.

Step S213: Charge the energy storager, and control, when it is determined that the energy storager is charged to a first preset threshold in the Nth light irradiating mode and the light irradiator is triggered, the light irradiator to irradiate first pulse light.

The controller 30 may detect and determine a charging status of the energy storager 10. For example, the controller 30 detects a current voltage of the energy storager 10, and compares the detected voltage with the first preset threshold to the Nth preset threshold, to determine whether the energy storager 10 is charged to any one of the first preset threshold to the Nth preset threshold in the Nth light irradiating mode. When the controller 30 determines that the energy storager 10 is charged to the first preset threshold in the Nth light irradiating mode, and the light irradiator 20 is triggered (that is, the light irradiator 20 meets a first trigger condition), the controller 30 controls the light irradiator 20 to irradiate the first pulse light. In some embodiments, that the light irradiator 20 meets the first trigger condition may include that a light outlet of the light irradiator 20 being in sufficient contact with the user's skin (which may be the light outlet being at least partially shielded by the user's skin) and a trigger button being triggered. That the light outlet of the light irradiator 20 being in sufficient contact with the user's skin may be determined by detecting a preset distance threshold by a distance detector disposed at the light outlet. That is, when the controller 30 determines that the energy storager 10 is charged to the first preset threshold, the light outlet of the light irradiator 20 is in sufficient contact with the user's skin, and the trigger button is triggered, the controller 30 controls the light irradiator 20 to irradiate the first pulse light. It may be understood that the distance detector is configured to detect contact or relative distance between the beauty instrument 100 and the skin. Based on the detection result of the distance detector, it can be determined that the light outlet of the light irradiator 20 is in sufficient contact with the user's skin. In this case, the distance detector detects the preset distance threshold, and the light irradiator 20 may irradiate the first pulse light on the skin of the user, to perform beauty and skincare treatment on the skin. It may be understood that the distance detector is a prior art, and may be but not limited to a photosensitive detector, an ultrasonic detector, a capacitance detector, a resistance detector, or the like. The working principle of detecting a preset threshold to determine whether it is in sufficient contact with the skin will not be described here.

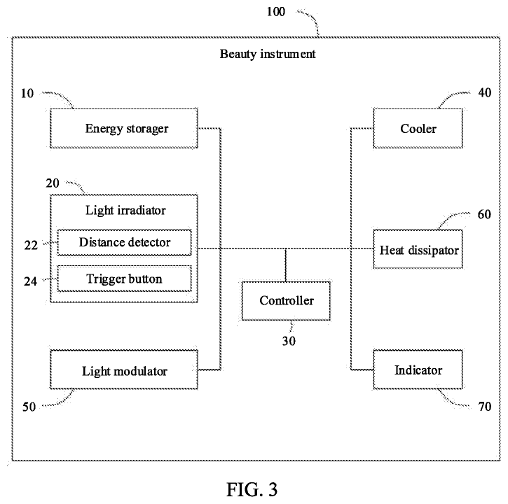

Referring to FIG. 3, in some embodiments, the light irradiator 20 may include a distance detector 22 and a trigger button 24. The light outlet may be an outlet used by the light irradiator 20 to irradiate light, and the light outlet may be further disposed at one end of a body of the beauty instrument 100. The distance detector 22 may be disposed on an end face of the light outlet, to detect distance information so as to determine, through detection, whether the light outlet is in sufficient contact with the user's skin. For example, when the user uses and operates the beauty instrument 100, the light outlet may be closely pressed against the skin of the user, and the distance detector 22 disposed at the light outlet may detect that a current distance is less than or equal to the preset distance threshold, so as to determine that the light outlet is in sufficient contact with the user's skin. When the light outlet is in sufficient contact with the user's skin, the pulse light irradiated by the light irradiator 20 by using the light outlet may directly reach the skin of the user without light leakage, thereby improving safety of the beauty instrument 100. The trigger button 24 may be disposed on the body of the beauty instrument 100, and is configured to receive an operation triggered by the user, for example, pressing or touching. In some embodiments, the trigger button 24 may be but is not limited to a physical button, a touch button, or the like. In response to the user's trigger operation, the corresponding trigger signal is transmitted to the controller 30, and the controller 30 determines that the trigger button 24 is triggered according to the trigger signal. It may be understood that the specific setting and working principle of trigger button 24 is the prior art, and will not be repeated here.

Step S214: Determine whether N times of irradiation of the pulse light have been completed.

The controller 30 determines whether the N times of irradiation of the pulse light have been completed. In some embodiments, the controller 30 determines, based on the Nth light irradiating mode configured for the beauty instrument 100, whether the N times of irradiation of the pulse light have been completed. For example, when the first light irradiating mode is configured for the beauty instrument 100, after the light irradiator 20 irradiates the first pulse light, the controller 30 may determine that one time of irradiation of the pulse light corresponding to the first light irradiating mode has been completed, and end a working procedure in the first light irradiating mode. When the Nth light irradiating mode is configured for the beauty instrument 100, after the light irradiator 20 irradiates the Nth pulse light, the controller 30 may determine that the N times of irradiation of the pulse light have been completed, and end a working procedure in the Nth light irradiating mode. When the controller 30 determines that the N times of irradiation of the pulse light have not been completed, step S215 may be performed in the method. In some embodiments, when the controller 30 determines that the N times of irradiation of the pulse light have been completed, the method may return to step S211.

Step S215: Continue to charge the energy storager, control, when it is determined that the energy storager is charged to a preset threshold corresponding to a next time of irradiation of the pulse light and the light irradiator is triggered, the light irradiator to irradiate next pulse light, and repeatedly perform the step of determining whether N times of irradiation of the pulse light have been completed.

In some embodiments, after the energy storager 10 is discharged to the light irradiator 20 to enable the light irradiator 20 to irradiate the pulse light, electric energy stored in the energy storager 10 is decreased by some or all of the electric energy. Only when the energy storager 10 continues to be charged to a next preset threshold, electric energy required by the light irradiator 20 for irradiating the next pulse light can be reached. When the light irradiator 20 completes irradiation of the next pulse light, the step of determining whether N times of irradiation of the pulse light have been completed is repeatedly performed. This process is cyclically performed until the N times of irradiation of the pulse light have been completed. In some embodiments, when the light irradiator 20 completes irradiation of the next pulse light, step S214 may be performed repeatedly to determine whether the N times of irradiation of the pulse light have been completed. This process is cyclically performed until the N times of irradiation of the pulse light have been completed.

When the controller 30 determines that the energy storager is charged to the Nth preset threshold and the light irradiator 20 is triggered (that is, the light irradiator 20 meets a second trigger condition), the light irradiator 20 is controlled to irradiate the Nth pulse light. In some embodiments, that the light irradiator 20 meets the second trigger condition may include that the light outlet of the light irradiator 20 is in sufficient contact with the user's skin (that is, the light irradiator 20 is at least partially shielded by the user's skin). That is, when the controller 30 determines that the energy storager 10 is charged to the Nth preset threshold and the distance detector detects the preset distance threshold, the controller 30 controls the light irradiator 20 to irradiate the Nth pulse light. It may be understood that the light outlet of the light irradiator 20 may be shielded at least partially or totally by the skin of the user. The light irradiator 20 may irradiate the Nth pulse light on the skin of the user, to perform beauty and skincare treatment on the skin.

In some embodiments, if the light irradiator 20 does not meet the second trigger condition, that is, it is determined by the controller 30 that the light outlet of the light irradiator 20 is not in sufficient contact with the user's skin based on the detection result of the distance detector 22, which means the distance detector 22 does not detect the preset distance threshold, light irradiation ends. For example, in a process in which the energy storager 10 is charged to the Nth preset threshold after the light irradiator 20 irradiates the (N−1)th pulse light, if the light irradiator 20 does not meet the second trigger condition, that is, it is determined by the controller 30 that the light outlet of the light irradiator 20 is not in sufficient contact with the user's skin based on the detection result of the distance detector 22 distance detector, for example, the light outlet is removed from the skin of the user so that the distance detector 22 fails to detect the preset distance threshold, light irradiation ends. Therefore, the light irradiator 20 is prevented from irradiating light when the light outlet is not in sufficient contact with the user's skin, so that light leakage can be avoided, and safety of the beauty instrument 100 is improved.

According to the control method for a beauty instrument in this solution, the energy storager 10 is charged at different stages based on the light irradiating mode configured for the beauty instrument 100. Therefore, the light irradiator 20 can irradiate the pulse light one time to N times based on charging at different stages, so that the beauty instrument 100 can provide various care manners for human skin in a plurality of light irradiating manners. This can meet a user's requirement for different functions of beauty and skincare, and improve function richness of the beauty instrument 100.

In addition, in the Nth light irradiating mode configured for the beauty instrument 100, when the energy storager 10 is charged to the first preset threshold, and the light irradiator 20 reaches the first trigger condition, that is, when the distance detector detects that the light outlet of the light irradiator 20 is in sufficient contact with the user's skin and the trigger button 24 is triggered, the energy storager 10 releases electric energy to the light irradiator 20, so that the light irradiator 20 irradiates the first pulse light. When the energy storager 10 is charged to the preset threshold corresponding to the next time of irradiation of the pulse light, and the light irradiator 20 meets the second trigger condition, that is, when the light outlet of the light irradiator 20 is in sufficient contact with the user's skin, the energy storager 10 releases electric energy to the light irradiator 20, so that the light irradiator 20 irradiates the next pulse light. This process is cyclically performed until the Nth pulse light is irradiated. In this way, when the user uses the beauty instrument 100 in the Nth light irradiating mode, the pulse light can be sequentially irradiated N times by operating the trigger button 24 only one time, so that an operation of the user is more convenient, and user experience is improved.

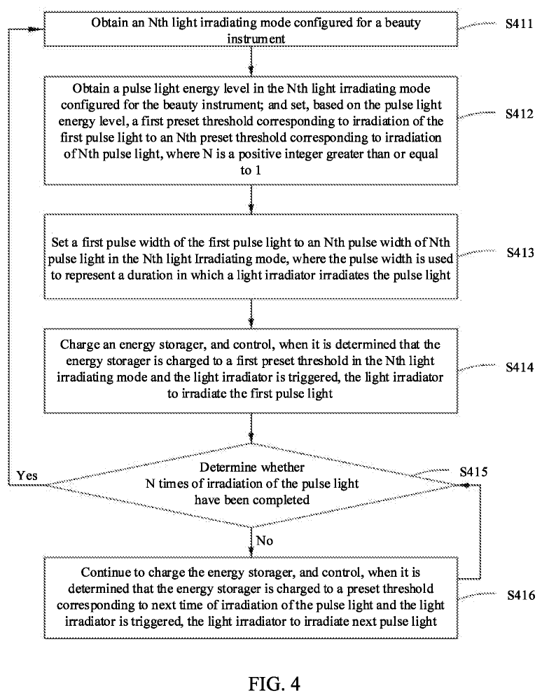

FIG. 4 shows a control method for a beauty instrument according to another embodiment of this application. For example, the control method shown in FIG. 4 may be performed by the beauty instrument 100 shown in FIG. 1 and FIG. 3, and includes the following steps.

Step S411: Obtain an Nth light irradiating mode configured for the beauty instrument.

For detailed description of step S411, refer to related descriptions of step S211 in FIG. 2, and FIG. 1 and FIG. 3. Details are not described herein again.

Step S412: Obtain a pulse light energy level in the Nth light irradiating mode configured for the beauty instrument, and set, based on the pulse light energy level, a first preset threshold corresponding to irradiation of the first pulse light to an Nth preset threshold corresponding to irradiation of Nth pulse light, where N is a positive integer greater than or equal to 1.

In some embodiments, the Nth light irradiating mode further includes the pulse light energy level.

After the controller 30 obtain the Nth light irradiating mode configured for the beauty instrument 100, the controller 30 may further obtain the pulse light energy level in the Nth light irradiating mode configured for the beauty instrument 100, and set, based on the pulse light energy level, the first preset threshold corresponding to irradiation of the first pulse light and the Nth preset threshold corresponding to irradiation of the Nth pulse light. In some embodiments, a preset threshold such as a pulse voltage threshold corresponding to irradiation of each pulse light may be set for each pulse light energy level in the Nth light irradiating mode. For example, a first level, a second level, a third level, . . . , and an Mth level may be set for the controller 30, where M is a positive integer greater than or equal to 1. Each level may correspond to a different degree of energy. It may be understood that, in different levels, the light irradiator 20 may irradiate pulse light with different energy. Correspondingly, different preset thresholds need to be set for the energy storager 10.

In some embodiments, when N is 1, the controller 30 obtains a pulse light energy level in the first light irradiating mode configured for the beauty instrument 100, and sets, based on the pulse light energy level, a first preset threshold corresponding to irradiation of the first pulse light.

In some embodiments, when N is a positive integer greater than or equal to 2, the controller 30 obtains the pulse light energy level in the Nth light irradiating mode configured for the beauty instrument 100, and sets, based on the pulse light energy level, the first preset threshold corresponding to irradiation of the first pulse light to the Nth preset threshold corresponding to irradiation of the Nth pulse light. It may be understood that when the Nth light irradiating mode is configured for the beauty instrument 100, the controller 30 may set the first preset threshold, the second preset threshold, . . . , the (N−1)th preset threshold, and the Nth preset threshold based on the pulse light energy level in the Nth light irradiating mode, so that a charging pulse voltage threshold or a charging time threshold required by the energy storager 10 to irradiate the pulse light N times in the Nth light irradiating mode can be set. In some embodiments, magnitudes of the first preset threshold to the Nth preset threshold are different, for example, may be incremented. For example, when N is three, the first preset threshold may be set to 100 V, the second preset threshold may be set to 150 V, and the third preset threshold may be set to 200 V.

In some embodiments, in the Nth light irradiating mode, when N is greater than or equal to 3, durations of time intervals between consecutive irradiations of the pulse light are different. For example, there is a first time interval between irradiation of (N−2)th pulse light and irradiation of the (N−1)th pulse light, and there is a second time interval between irradiation of the (N−1)th pulse light and irradiation of the Nth pulse light. A duration of the first time interval is different from a duration of the second time interval. That is, there is a time interval between two times of adjacent irradiation of the pulse light, and durations of two adjacent time intervals are different. For example, when N is 3, that is, in the third light irradiating mode, there is a first time interval t1 between irradiation of the first pulse light and irradiation of the second pulse light, and there is a second time interval t2 between irradiation of the second pulse light and irradiation of the third pulse light, where a duration of the first time interval t1 is different from a duration of the second time interval t2. In some embodiments, the time interval may be charging time of the energy storager 10. Because magnitudes of the first preset threshold to the Nth preset threshold are different, charging durations of the energy storager 10 are different, and therefore time intervals between consecutive irradiation of the pulse light are also different.

Step S413: Set a first pulse width of the first pulse light to an Nth pulse width of Nth pulse light in the Nth light irradiating mode, where the pulse width is used to represent a duration in which the light irradiator irradiates the pulse light.

In some embodiments, the controller 30 may further set pulse widths corresponding to the pulse light irradiated N times in the Nth light irradiating mode. For example, when N is greater than or equal to 3, the controller 30 may set a first pulse width of the first pulse light irradiated by the light irradiator 20, a second pulse width of the second pulse light irradiated by the light irradiator, . . . , and an Nth pulse width of the Nth pulse light irradiated by the light irradiator. The first pulse width is used to represent a duration in which the light irradiator 20 irradiates the first pulse light, the second pulse width is used to represent a duration in which the light irradiator 20 irradiates the second pulse light, . . . , and the Nth pulse width is used to represent a duration in which the light irradiator 20 irradiates the Nth pulse light. For example, when N is 3, that is, in the third light irradiating mode, the controller 30 may set the first pulse width to 0.2 millisecond, set the second pulse width to 0.5 millisecond, and set the third pulse width to 0.8 millisecond, that is, set a duration in which the light irradiator 20 irradiates the first pulse light to 0.2 millisecond, set a duration in which the light irradiator 20 irradiates the second pulse light to 0.5 millisecond, and set a duration in which the light irradiator 20 irradiates the third pulse light to 0.8 millisecond.

It may be understood that a sequence of performing steps S412 and S413 may be interchanged, or the two steps may be performed simultaneously, or may be combined into one step for execution. This is not limited in this application.

Step S414: Charge the energy storager, and control, when it is determined that the energy storager is charged to a first preset threshold in the Nth light irradiating mode and the light irradiator is triggered, the light irradiator to irradiate first pulse light.

For detailed description of step S414, refer to related descriptions of step S213 in FIG. 2, and FIG. 1 and FIG. 3. Details are not described herein again.

Step S415: Determine whether N times of irradiation of the pulse light have been completed.

For detailed description of step S415, refer to related descriptions of step S214 in FIG. 2, and FIG. 1 and FIG. 3. Details are not described herein again.

In some embodiments, when the controller 30 determines that the N times of irradiation of the pulse light have been completed, the method may return to step S411. When the controller 30 determines that the N times of irradiation of the pulse light have not been completed, step S416 may be performed in the method.

Step S416: Continue to charge the energy storager, control, when it is determined that the energy storager is charged to a preset threshold corresponding to a next time of irradiation of the pulse light and the light irradiator is triggered, the light irradiator to irradiate next pulse light, and repeatedly perform the step of determining whether N times of irradiation of the pulse light have been completed.

For detailed description of step S416, refer to related descriptions of step S215 in FIG. 2, and FIG. 1 and FIG. 3. Details are not described herein again.

Referring to FIG. 3, the beauty instrument 100 may further include a cooler 40.

The cooler 40 may be configured to reduce a temperature. In some embodiments, the cooler 40 may be disposed around the light outlet of the light irradiator 20. When the light outlet is closely pressed against to the skin of the user, the cooler 40 is also closely pressed against to the skin of the user. When the light irradiator 20 irradiates the pulse light to the skin of the user, energy of the pulse light may generate specific heat. The cooler 40 may cool and reduce a temperature of the skin of the user, so as to offset the heat generated by the energy of the pulse light, thereby improving comfort of the user when using the beauty instrument 100.

The cooler 40 may work at different power to produce different cooling effects. In some embodiments, the working mode of the beauty instrument 100 may further include an ice compress mode. When the ice compress mode is configured for the beauty instrument 100, the cooler 40 works at first power. When the Nth light irradiating mode is configured for the beauty instrument 100, the cooler 40 works at second power. The second power is less than the first power, that is, cooling intensity of the cooler 40 in the ice compress mode is greater than cooling intensity thereof in the Nth light irradiating mode. The cooler 40 may generate large cooling intensity in the ice compress mode, thereby implementing a caring effect such as ice compress or cold therapy on the skin of the user. In some embodiments, the cooler 40 may include a semiconductor cooling component and a heat conductive component, where the heat conductive component may be but is not limited to a sapphire, glass, or the like.

Referring to FIG. 3, the beauty instrument 100 may further include a light modulator 50. The light modulator 50 is configured to irradiate light of different colors to assist the beauty instrument 100 in different working modes. Colored light irradiated by the light modulator 50 and the pulse light irradiated by the light irradiator 20 may be approximately in a same direction, and may be irradiated to the skin of the user together. In some embodiments, when the Nth light irradiating mode is configured for the beauty instrument 100, and the light irradiator 20 irradiates the first pulse light to the Nth pulse light, the light modulator 50 may respectively irradiate light of a first color to light of an Nth color. When the ice compress mode is configured for the beauty instrument 100, the light modulator 50 may irradiate light of an (N+1)th color. The first color, the Nth color, and the (N+1)th color are different. In some embodiments, the light modulator 50 may be but is not limited to an LED or the like. For example, when the Nth light irradiating mode is configured for the beauty instrument 100, and the light irradiator 20 irradiates the first pulse light, the light modulator 50 may irradiate red light, so as to assist the first pulse light in achieving beauty and skincare effects such as skin whitening and spot-reducing. When the Nth light irradiating mode is configured for the beauty instrument 100, and the light irradiator 20 irradiates the second pulse light, the light modulator 50 may irradiate yellow light, so as to assist the second pulse light in achieving beauty and skincare effects such as removing redness and acne mark. When the Nth light irradiating mode is configured for the beauty instrument 100, and the light irradiator 20 irradiates the third pulse light, the light modulator 50 may irradiate red light and yellow light, so as to assist the third pulse light in achieving beauty and skincare effects such as skin rejuvenation and brightening. When the ice compress mode is configured for the beauty instrument 100, the light modulator 50 may irradiate pale yellow light to assist the ice compress mode in achieving beauty and skincare effects such as ice compress and relaxation.

Referring to FIG. 3, the beauty instrument 100 may further include a heat dissipator 60 and an indicator 70.

The heat dissipator 60 is configured to dissipate heat when the beauty instrument 100 works. It may be understood that when the beauty instrument 100 works, for related modules such as the energy storager 10 (which is charged and performs discharging), the light irradiator 20 (which irradiates the pulse light), the controller 30 (which works), the cooler 40 (which performs cooling), and the light modulator 50 (which irradiates light), because the related modules generate some heat in the body of the beauty instrument 100, the heat dissipator 60 may be configured to: guide the heat in the body of the beauty instrument 100 to the outside of the body, so as to avoid overheating of the beauty instrument 100, and improve use safety of the beauty instrument 100. In some embodiments, the heat dissipator 60 and the light outlet of the light irradiator 20 may be separately disposed on two opposite ends of the beauty instrument 100. The light outlet may be at an end placed close to the skin of the user, and the heat dissipator 60 may be at an end far away from the skin of the user, so that the dissipated heat does not affect the user, and user experience is improved. It may be understood that when the beauty instrument 100 is powered on, the heat dissipator 60 may simultaneously start working, and synchronously dissipate heat. When the beauty instrument 100 is powered off, the heat dissipator 60 may stop working.

The indicator 70 is configured to indicate different working modes of the beauty instrument 100. In some embodiments, the indicator 70 may be but is not limited to a display screen, an LED indicator, or the like, and may differentially indicate a current working mode of the beauty instrument 100. When the Nth light irradiating mode or the ice compress mode is configured for the beauty instrument, the indicator 70 may separately indicate different modes. For example, when N is 1, that is, when the first light irradiating mode is configured for the beauty instrument 100, the indicator 70 may display first indication information. When the Nth light irradiating mode is configured for the beauty instrument 100, the indicator 70 may display Nth indication information. When the ice compress mode is configured for the beauty instrument 100, the indicator 70 may display (N+1)th indication information. The indication information may be but is not limited to text or image information on the display screen, on/off or color information of the LED light, or the like, so that different indication information is used to indicate a current working mode of the beauty instrument 100, thereby facilitating user observation.

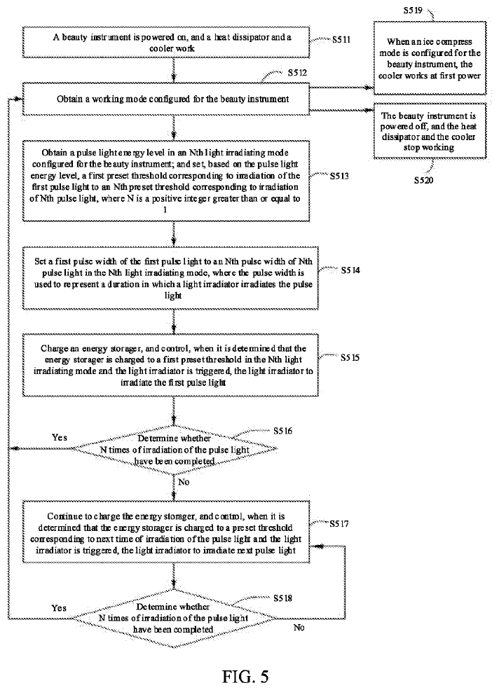

FIG. 5 shows a control method for a beauty instrument according to another embodiment of this application. For example, the control method shown in FIG. 5 may be performed by the beauty instrument 100 shown in FIG. 3, and includes the following steps.

Step S511: The beauty instrument is powered on, and the heat dissipator and the cooler work.

In some embodiments, after the beauty instrument 100 is powered on, the heat dissipator 60 and the cooler 40 may be started accordingly. The heat dissipator 60 works to dissipate heat for the beauty instrument 100, and the cooler 40 may work at second power.

In some other embodiments, if the beauty instrument 100 is powered on for the first time, a step of initializing system data may be further included.

Step S512: Obtain a working mode configured for the beauty instrument.

In some embodiments, a user may configure the working mode of the beauty instrument 100 through operation, and the controller 30 obtains the working mode configured for the beauty instrument 100. In some embodiments, the working mode configured for the beauty instrument 100 may include an Nth light irradiating mode or an ice compress mode.

For detailed description of step S512, refer to related descriptions of step S211 in FIG. 2, and FIG. 1 and FIG. 3. Details are not described herein again.

Step S513: Obtain a pulse light energy level in the Nth light irradiating mode configured for the beauty instrument; and set, based on the pulse light energy level, a first preset threshold corresponding to irradiation of the first pulse light to an Nth preset threshold corresponding to irradiation of Nth pulse light, where N is a positive integer greater than or equal to 1.

For detailed description of step S513, refer to related descriptions of step S212 in FIG. 2, step S412 in FIG. 4, and FIG. 1 and FIG. 3. Details are not described herein again.

In some embodiments, when N is 1, that is, when a first light irradiating mode is configured for the beauty instrument, the indicator 70 may display first indication information to indicate that the first light irradiating mode is currently configured for the beauty instrument 100. When an Nth light irradiating mode is configured for the beauty instrument, the indicator 70 may display Nth indication information to indicate that the Nth light irradiating mode is currently configured for the beauty instrument 100.

Step S514: Set a first pulse width of the first pulse light to an Nth pulse width of Nth pulse light in the Nth light irradiating mode, where the pulse width is used to represent a duration in which the light irradiator irradiates the pulse light.

For detailed description of step S514, refer to related descriptions of step S413 in FIG. 4, and FIG. 1 and FIG. 3. Details are not described herein again.

It may be understood that sequences of performing steps S513 and S514 may be interchanged, or the two steps may be performed simultaneously, or may be combined into one step for execution. This is not limited in this application. In some other embodiments, steps S513 and S514 may be replaced with related descriptions of step S212 in FIG. 2, and FIG. 1 and FIG. 3.

Step S515: Charge the energy storager, and control, when it is determined that the energy storager is charged to a first preset threshold in the Nth light irradiating mode and the light irradiator is triggered, the light irradiator to irradiate first pulse light.

For detailed description of step S515, refer to related descriptions of step S213 in FIG. 2, and FIG. 1 and FIG. 3. Details are not described herein again.

In some embodiments, when N is 1, that is, when the first light irradiating mode is configured for the beauty instrument 100, and the light irradiator 20 irradiates the first pulse light, the light modulator 50 may simultaneously irradiate light of a first color. It may be understood that both time of irradiating the first pulse light by the light irradiator 20 and time of irradiating the light of the first color by the light modulator 50 are a first pulse width.

In some embodiments, when the Nth light irradiating mode is configured for the beauty instrument 100, and the light irradiator 20 irradiates the first pulse light, the light modulator 50 may simultaneously irradiate light of an Nth color. It may be understood that both time of irradiating the first pulse light by the light irradiator 20 and time of irradiating the light of the Nth color by the light modulator 50 are a first pulse width.

It may be understood that, when the energy storager 10 is not charged to the first preset threshold or the light irradiator 20 is not triggered, this step continues to be performed. When the light irradiator 20 is triggered, the light irradiator 20 irradiates the first pulse light.

Step S516: Determine whether N times of irradiation of the pulse light have been completed.

For detailed description of step S516, refer to related descriptions of step S214 in FIG. 2, and FIG. 1 and FIG. 3. Details are not described herein again.

In some embodiments, when the controller 30 determines that the N times of irradiation of the pulse light have been completed, the method may return to step S512. When the controller 30 determines that the N times of irradiation of the pulse light have not been completed, step S517 may be performed in the method.

Step S517: Continue to charge the energy storager, and control, when it is determined that the energy storager is charged to a preset threshold corresponding to a next time of irradiation of the pulse light and the light irradiator is triggered, the light irradiator to irradiate next pulse light.

For detailed description of step S517, refer to related descriptions of step S215 in FIG. 2, and FIG. 1 and FIG. 3. Details are not described herein again.

In some embodiments, when the light irradiator 20 irradiates the Nth pulse light, the light modulator 50 may simultaneously irradiate the light of the Nth color. It may be understood that both time of irradiating the Nth pulse light by the light irradiator 20 and time of irradiating the light of the Nth color by the light modulator 50 are an Nth pulse width.

It may be understood that, when the energy storager 10 is not charged to the Nth preset threshold or the light irradiator 20 is not triggered, this step continues to be performed. When the light irradiator 20 is triggered, the light irradiator 20 irradiates the Nth pulse light.

Step S518: Determine whether N times of irradiation of the pulse light have been completed.

For detailed description of step S518, refer to related descriptions of step S214 in FIG. 2, and FIG. 1 and FIG. 3. Details are not described herein again.

In some embodiments, when the controller 30 determines that the N times of irradiation of the pulse light have been completed, the method may return to step S512. When the controller 30 determines that the N times of irradiation of the pulse light have not been completed, step S517 may continue to be performed in the method.

Step S519: When the ice compress mode is configured for the beauty instrument, the cooler works at first power.

In some embodiments, when the ice compress mode is configured for the beauty instrument 100, the cooler 40 switches from working at the second power to working at the first power. Because the first power is greater than the second power, the cooler 40 has larger cooling strength. In addition, the light modulator 50 may irradiate light of an (N+1)th color to implement effects such as ice compress and relaxation on the skin of the user.

In addition, the indicator 70 may display (N+1)th indication information to indicate that the ice compress mode is currently configured for the beauty instrument 100.

It may be understood that when the ice compress mode is configured for the beauty instrument 100, a duration of the ice compress mode may be not limited, and the working mode of the beauty instrument 100 may be switched by receiving an operation of the user, that is, the method procedure may return to step S512 after step S519 is performed.

It may be understood that when the method returns to step S512 from step S516, step S518, and step S519, the indicator 70 may stop displaying the indication information.

Step S520: The beauty instrument is powered off, and the heat dissipator and the cooler stop working.

In some embodiments, the heat dissipator 60 and the cooler 40 may stop working when the beauty instrument 100 is powered off.

According to the control method for a beauty instrument in this solution, based on the Nth light irradiating mode or the ice compress mode configured for the beauty instrument 100, the light irradiator 20 may irradiate the pulse light one time to N times based on charging at different stages, or work based on different power of the cooler 40, and the light modulator 50 irradiate the colored light. Therefore, the beauty instrument 100 can provide various care manners for human skin based on a plurality of working modes. This can meet a user's requirement for different functions of beauty and skincare, and improve function richness of the beauty instrument 100.

The foregoing descriptions about implementations allow a person skilled in the art to understand that, for the purpose of convenient and brief description, division of the foregoing function modules is taken as an example for illustration. In actual application, the foregoing functions can be allocated to different modules and implemented according to a requirement, that is, an inner structure of an apparatus is divided into different function modules to implement all or some of the functions described above.

In the several embodiments provided in this application, it should be understood that the disclosed system may be implemented in another manner. For example, the described system embodiment is merely an example. For example, the module or unit division is merely logical function division and may be other division in actual implementation. For example, a plurality of units or components may be combined or integrated into another system, or some features may be ignored or not performed. In addition, the displayed or discussed mutual couplings or direct couplings or communication connections may be implemented through some interfaces. The indirect couplings or communication connections between the apparatuses or units may be implemented in electronic, mechanical, or other forms.

For a person skilled in the art, it is clear that this application is not limited to the details of the foregoing example embodiments. Instead, this application can be implemented in another specific form without departing from the spirit or the essential feature of this application. Therefore, appropriate modifications and changes made to the foregoing embodiments should fall within the protection scope of this application, provided that they are within the essence and spirit of this application.

What is claimed is:

1. A control method for a beauty instrument, wherein the beauty instrument comprises an energy storager and a light irradiator, the energy storager is configured to be charged and perform discharging, the light irradiator is configured to irradiate pulse light based on power supply of the energy storager, and the method comprises:
obtaining an Nth light irradiating mode configured for the beauty instrument;
setting, based on the Nth light irradiating mode, the light irradiator to irradiate the pulse light N times and a preset threshold corresponding to each time of irradiation of the pulse light, wherein N is a positive integer greater than or equal to 1;
charging the energy storager, and controlling, when it is determined that the energy storager is charged to a first preset threshold of the Nth light irradiating mode and the light irradiator is triggered, the light irradiator to irradiate first pulse light;
determining whether N times of irradiation of the pulse light have been completed, if no, continuing to charge the energy storager, and controlling, when it is determined that the energy storager is charged to a preset threshold corresponding to a next time of irradiation of the pulse light and the light irradiator is triggered, the light irradiator to irradiate next pulse light; and
repeatedly performing the step of determining whether N times of irradiation of the pulse light have been completed;
wherein,
the beauty instrument further comprises a light modulator, configured to irradiate light of different colors, and the method further comprises:
in the Nth light irradiating mode, controlling the light modulator to respectively irradiate light of a first color to light of an Nth color when the first pulse light to Nth pulse light are irradiated; or
the beauty instrument further comprises a cooler and a light modulator configured to irradiate light of different colors, and the method further comprises:
when an ice compress mode is configured for the beauty instrument, controlling the light modulator to irradiate light of an (N+1)th color, wherein the first color, the Nth color, and the (N+1)th are different.

2. The method according to claim 1, wherein the preset threshold is a pulse voltage threshold or a charging time threshold.

3. The method according to claim 1, wherein the Nth light irradiating mode further comprises a pulse light energy level, and the method further comprises:
obtaining the pulse light energy level in the Nth light irradiating mode configured for the beauty instrument; and
setting, based on the pulse light energy level, the first preset threshold corresponding to irradiation of the first pulse light to an Nth preset threshold corresponding to irradiation of Nth pulse light.

4. The method according to claim 1, wherein when N is greater than or equal to three, durations of time intervals between consecutive irradiations of the pulse light are different in the Nth light irradiating mode.

5. The method according to claim 1, wherein the method further comprises: setting a first pulse width of the first pulse light to an Nth pulse width of Nth pulse light in the Nth light irradiating mode, wherein the pulse width is used to represent a duration in which the light irradiator irradiates the pulse light.

6. The method according to claim 1, wherein when the beauty instrument further comprises the cooler, the method further comprises:
when the ice compress mode is configured for the beauty instrument, enabling the cooler to work at first power.

7. The method according to claim 6, wherein the method further comprises:
when the light irradiating mode is configured for the beauty instrument, enabling the cooler to work at second power, wherein the second power is less than the first power.

8. (canceled)

9. The method according to claim 1, wherein the beauty instrument further comprises an indicator, and the method further comprises:
when the Nth light irradiating mode or the ice compress mode is configured for the beauty instrument, enabling the indicator to separately indicate different modes.

10. The method according to claim 1, wherein the beauty instrument further comprises a distance detector and a trigger button;
the controlling, when it is determined that the energy storager is charged to a first preset threshold in the Nth light irradiating mode and the light irradiator is triggered, the light irradiator to irradiate first pulse light comprises: controlling, when it is determined that the energy storager is charged to the first preset threshold in the Nth light irradiating mode, the distance detector of the beauty instrument detects a preset distance threshold, and the trigger button is triggered, the light irradiator to irradiate the first pulse light; and
the controlling, when it is determined that the energy storager is charged to a preset threshold corresponding to a next time of irradiation of the pulse light and the light irradiator is triggered, the light irradiator to irradiate next pulse light comprises: controlling, when it is determined that the energy storager is charged to the preset threshold corresponding to the next time of irradiation of the pulse light and the distance detector of the beauty instrument detects the preset distance threshold, the light irradiator to irradiate the next pulse light.

11. (canceled)
